(19) United States
(12) Patent Application Publication
(10) Pub. No.: US 2006/0160134 A1
(43) Pub. Date: Jul. 20, 2006

(54) Novel application of biosensors for diagnosis and treatment of disease

(76) Inventors: Richard J. Melker, Gainesville, FL (US); Donn Michael Dennis, Gainesville, FL (US); Charles R. Martin, Gainesville, FL (US); Jon D. Stewart, Gainesville, FL (US)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/296,757

(22) Filed: Dec. 7, 2005

Related U.S. Application Data
(63) Continuation-in-part of application No. 10/345,532, filed on Jan. 16, 2003, now Pat. No. 6,974,706.
(63) Continuation-in-part of application No. 10/274,829, filed on Oct. 21, 2002.
(63) Continuation-in-part of application No. 10/744,789, filed on Dec. 23, 2003.

(51) Int. Cl. G01N 33/53

(52) U.S. Cl. 435/7.1

(57) ABSTRACT

A method for detecting compounds of interest in bodily fluids, including exhaled breath and blood. The present invention uses biosensors that mimic naturally occurring cellular mechanisms, including RNA oligonucleotide chains or “aptamers,” in combination with signaling agents or nanotechnology to provide an effective and efficient method for diagnosing a condition and/or disease within a patient. The subject invention also provides a method for screening those analytes/biomarkers likely to be present in exhaled breath.

### CROSS-REFERENCE TO RELATED APPLICATION

This application is a continuation-in-part of co-pending U.S. patent application Ser. No. 10/345,532, filed Jan. 16, 2003; Ser. No. 10/274,829, filed Oct. 21, 2002; and Ser. No. 10/744,789, filed Dec. 23, 2003, all of which are hereby incorporated by reference herein in their entirety, including any figures, tables, or drawings.

### GOVERNMENT SUPPORT

The subject matter of this application has been supported by a research grant from the National Science Foundation (Grant Number NSF: EEC 02-10580). Accordingly, the government may have certain rights in this invention.

### BACKGROUND OF INVENTION

There is a great need for the development of efficient and accurate methods related to the detection and identification of chemical and biological agents (hereinafter “analyte”) including, but not limited to, nucleic acids, proteins, illicit drugs, explosives, toxins, pharmaceuticals, carcinogens, poisons, allergens, and infectious agents. Current methods of detecting analytes require extraction of a sample into organic solvents, followed by analysis using stand alone analytical systems such as gas-liquid chromatography and/or mass spectroscopy. These methods are time-consuming and often expensive. The development of a biosensor device that could accurately and efficiently detect/screen for chemical and biological agents would therefore provide a significant cost and time benefit.

Three recent advancements in medicine are particularly germane to expanding the potential of detecting analytes, in particular with regard to the diagnosis and treatment of disease: nanotechnology, biodetectors (biosensors), and the identification of biomarkers for specific diseases and/or conditions. Nanotechnology, in particular nanoparticles, offers many advantages when used for applications such as the delivery of bioactive agents (i.e., DNA, AIDS drugs, gene therapy, immunosuppressants, chemotherapeutics), and drug uptake and degradation (i.e., enzyme encapsulation). For example, nanoparticles have been proposed as providing site-specific distribution of drugs to, and minimization of loss from, a target site. Appropriately sized particles have been proposed wherein such particles can be delivered to selected tissues to release their drug load in a controlled and sustained manner.

The term “biodetectors” or “biosensors” relates to the use of naturally occurring and synthetic compounds as highly specific and extraordinarily sensitive detectors of various types of molecules and markers of disease. Biosensor manufacture mimics the naturally occurring mechanisms of DNA, RNA, and protein synthesis in cells.

Aptamers have recently been identified as potentially effective biosensors for molecules and compounds of scientific and commercial interest (see Brody, E. N. and L. Gold, “Aptamers as therapeutic and diagnostic agents,” J. Biotechnol., 74(1):5-13 (2000) and Brody et al., “The use of aptamers in large arrays for molecular diagnostics,” Mol. Diagn., 4(4):381-8 (1999)). For example, aptamers have demonstrated greater specificity and robustness than antibody-based diagnostic technologies. In contrast to antibodies, whose identification and production completely rest on animals and/or cultured cells, both the identification and production of aptamers takes place in vitro without any requirement for animals or cells. Aptamer synthesis is far cheaper and reproducible than antibody-based diagnostic tests. Aptamers are produced by solid phase chemical synthesis, an accurate and reproducible process with consistency among production batches. An aptamer can be produced in large quantities by polymerase chain reaction (PCR) and once the sequence is known, can be assembled from individual naturally occurring nucleotides and/or synthetic nucleotides. Aptamers are stable to long-term storage at room temperature, and, if denatured, aptamers can easily be renatured, a feature not shared by antibodies. Furthermore, aptamers have the potential to measure concentrations of ligand in orders of magnitude lower (parts per trillion or even quadrillion) than those antibody-based diagnostic tests. These inherent characteristics of aptamers make them attractive for diagnostic applications.

A number of “molecular beacons” (often fluorescence compounds) can be attached to aptamers to provide a means for signaling the presence of and quantifying a target analyte. For instance, an aptamer specific for cocaine has recently been synthesized (Stojanovic, M. N. et al., “Aptamer-based folding fluorescent sensor for cocaine,” J. Am. Chem. Soc., 123(21):4928:31 (2001)). A fluorescence beacon, which quenches when cocaine is reversibly bound to the aptamer is used with a photodetector to quantify the concentration of cocaine present. Aptamer-based biosensors can be used repeatedly, in contrast to antibody-based tests that can be used only once.

Of particular interest as a beacon are amplifying fluorescent polymers (AFP). AFPs with a high specificity to TNT and DNT have been developed. Interestingly, a detector based on AFP technology also detects propofol, an intravenous anesthetic agent, in extremely low concentration. The combination of AFP and aptamer technologies holds the promise of robust, reusable biosensors that can detect compounds in minute concentrations with high specificity.

The term “biomarker” refers to a specific biochemical in the body that has a particular molecular feature to make it useful for diagnosing and measuring the progress of disease or the effects of treatment. For example, common metabolites or biomarkers found in a person's breath, and the respective diagnostic condition of the person providing such metabolite include, but are not limited to, acetaldehyde (source: ethanol, X-threonine; diagnosis: intoxication), acetone (source: acetoacetate; diagnosis: diet/diabetes), ammonia (source: deamination of amino acids; diagnosis: uremia and liver disease), CO (carbon monoxide) (source: CH₂Cl₂, elevated % COHb; diagnosis: indoor air pollution), chloroform (source: halogenated compounds), dichlorobenzene (source: halogenated compounds), diethylamine (source: choline; diagnosis: intestinal bacterial overgrowth), H (hydrogen) (source: intestines; diagnosis: lactose intolerance), isoprene (source: fatty acid; diagnosis: metabolic stress), methanethiol (source: methionine; diagnosis: intestinal bacterial overgrowth), methylethylketone (source: fatty acid; diagnosis: indoor air pollution/diet), O-toluidine (source: carcinoma metabolite; diagnosis: bronchogenic carcinoma), pentane sulfides and sulfides (source: lipid peroxidation; diagnosis: myocardial infarction), H₂S (source: metabolism; diagnosis: periodontal disease/ovulation), MeS (source: metabolism; diagnosis: cirrhosis), and Me₂S (source: infection; diagnosis: trench mouth).

Mechanisms of drug metabolism are extremely complex and are influenced by a number of factors including competitive binding on protein and red blood cells with other molecules, enzymatic activity, particularly in the liver, protein, and red blood cell concentration and a myriad of other factors. Exhaled breath holds the promise of a diagnostic technique, which can measure drug concentration real-time and thereby allow convenient determination of pharmacokinetics and pharmacodynamics of multiple compounds in real-time.

Accordingly, there are a number of medical conditions that can be monitored by detecting and/or measuring biomarkers present in a person's breath and other bodily fluids. While there has been technology generated towards the synthesis and use of aptamers and other multimolecular devices as biosensors, there exists little technology that address the use of exhaled breath in conjunction with apatmers as biosensors for the diagnosis and treatment of disease or as detectors for a wide range of naturally occurring and synthetic compounds. It is therefore desirable to provide a low-cost means for accurately and timely detecting and/or measuring the presence of metabolites in a person's bodily fluids in low concentrations via non-invasive methods. Further, in order to effectively apply exhaled breath sensing technology, there is a pressing need for an efficient screening method for determining which analytes/biomarkers are likely to be detectable in exhaled breath.

### BRIEF SUMMARY

The present invention provides unique methods for detecting analytes/biomarkers of interest in bodily fluids. The invention utilizes molecular recognition agents in combination with a signaling agent (such as a molecular beacon, fluorescent polymer, or volatile biomarker) to non-invasively detect drugs, biomarkers, and other analytes in extremely low concentrations in exhaled breath and other bodily fluids. In a preferred embodiment, the molecular recognition agent is an aptamer, a highly specific nucleic acid-based ligand, that highly specific for a target analyte/biomarker.

In one embodiment, the systems of the invention include aptamers attached to signaling agents to provide a means for detecting, signaling, and/or quantifying virtually any compound of interest in exhaled breath.

In another embodiment, the systems of the invention include aptamers in combination with nanotechnology (i.e., nanotubes) to provide an effective method for signaling the presence of a target analyte in bodily fluids, including but not limited to the exhaled breath and blood.

In one embodiment, the present invention provides a method for analyzing analytes/biomarkers in exhaled breath using a volatile or “surrogate” biomarker. For example, one method for analyzing analytes/biomarkers in bodily fluids comprises the use of an aptamer or other form of molecular recognition agent attached directly to a volatile or “surrogate” biomarker. Volatile or “surrogate” biomarkers include substances and compounds that can be detected in bodily fluids by various sensing means. In certain embodiments, the surrogate biomarkers become volatile after the molecular recognition agent binds to the specific/target biomarker.

In another embodiment, the present invention provides a method for analyzing analytes/biomarkers in exhaled breath using an aptamer or other form of molecular recognition agent attached to a volatile biomarker that contains an amplifying fluorescent polymer (AFP). In an alternate, related embodiment, the invention provides a method for analyzing analytes/biomarkers in exhaled breath using an aptamer or other molecular recognition agent attached directly to an AFP.

According to the subject invention, surrogate biomarkers, molecular beacons, and/or amplifying fluorescent polymers can be detected and/quantified using conventional sensing devices. For example, previously disclosed sensor technology such as, but not limited to, microgravimetric sensor technology, electrochemical sensor technology, gas chromatography/mass spectrometry sensor technology, infrared spectroscopy technology, ion mobility spectrometry, photo-ionization sensor technology, thickness-shear mode sensor technology, amplifying fluorescent polymer sensor technology, fiber optic microsphere sensor technology, semiconductor sensor technology, conductive polymer sensor technology, surface acoustic wave sensor technology, immunoassays, interdigitated microelectrode array sensor technology, microelectromechanical sensor technology, and molecularly imprinted polymeric film sensor technology, can be used in accordance with the subject invention to detect and/or quantify the target analytes/biomarkers in bodily fluids.

In another embodiment, the present invention provides a method for analyzing analytes/biomarkers in bodily fluids, including blood, using a biosensor that includes a nanotube and an aptamer or other molecular recognition agent. The nanotube comprises a hollow tubular body defining an inner void, comprising a first end and a second end, and a volatile or “surrogate” biomarker contained within the hollow tubular body. In a preferred embodiment, the first end of the tubular body is open and a first end cap bound to an aptamer or other molecular recognition agent is positioned over the first open end to close the first end. The second end of the tube is closed or similarly capped as the first end.

According to the present invention, nanotubes containing volatile or “surrogate” biomarkers are provided that release the volatile or “surrogate” biomarkers from the nanotube under a variety of conditions to diagnose and/or treat a disease. In a preferred embodiment, an aptamer is designed for a biomarker of prostate cancer. Prostate cancers produce a protein, prostate specific antigen (PSA). An aptamer could be designed that is specific for PSA (PSA-aptamer). The PSA-aptamer can be attached to an end cap that fits on the end of a nanotube. A rapid test for the presence of prostate cancer, or a recurrence, could be developed where the volatile or “surrogate” biomarker is released from the nanotube after PSA (the biomarker of interest) interacts with the PSA-aptamer and “uncaps” the nanotubes. Using any of a number of previously disclosed detector technologies, the volatile biomarker is detected in exhaled breath, which indicates the presence of PSA in the blood.

Biosensing exhaled breath utilizing methods disclosed herein can be applied to a wide range of point of care (POC) diagnostic tests. For example, potential applications include detection of licit and illicit drugs, detection of a wide range of biomarkers related to specific diseases, and detection of any other compounds that appear in blood or other bodily fluids. These tests can be highly quantitative with the quantity of volatile or “surrogate” biomarker released/detectable being proportional to the quantity of a target compound in a sample of bodily fluid.

Moreover, exhaled breath detection using the method of the present invention can evaluate the efficacy of interventions in real-time. For example, it is known that isoprostane levels increase in cerebral spinal fluid and blood after traumatic brain injury. If isoprostane is readily detectable in exhaled breath by using an isoprostane specific biosensor according to the present invention, it can be possible to evaluate the efficacy of interventions in real-time for treating traumatic brain injury. In addition, the method of the present invention can also evaluate pharmacodynamics and pharmacokinetics for drug interventions in individuals.

The present invention also provides an effective and efficient method for screening analytes/biomarkers likely to be detectable in exhaled breath. Presently, it is unclear how often and to what extent disease specific biomarkers are present in exhaled breath. An embodiment of the present invention includes a screening process employing human blood placed in small vials to provide a cost-effective means to screen a wide variety of samples in conjunction with standard diagnostic equipment.

In a preferred embodiment, biomarkers detectable in exhaled breath are screened by the following steps: (1) providing samples of human blood free of potential biomarkers or including potential biomarkers, (2) incubating the blood samples at body temperature, and (3) measuring the concentration of a biomarker in whole blood, plasma, an ultrafiltrate, and/or in headspace. Preferably, a target biomarker is added to vials containing a small amount of blood in concentrations in the range likely to be found in vivo. The sample vials are incubated at body temperature and the concentration of the target biomarker in whole blood, plasma, in an ultrafiltrate, and in headspace are measured using conventional quantitative devices, such as LC-MS (liquid chromatography-mass spectroscopy) which is capable of measuring concentrations in parts per trillion. Free biomarkers/analytes (in ultrafiltrate) should be in equilibrium with biomarkers/analytes present in headspace. Target biomarkers present in headspace can be identified as those likely to be present in exhaled breath. The screening methodology according to the subject invention enables the production of a vast library of drugs, biomarkers, and other analytes likely to be present in bodily fluids.

In another embodiment, the screening method according to the present invention can include providing blood specimens from patients with known diseases (i.e., Alzheimer's disease, multiple sclerosis) and screening the specimens for the presence of biomarkers in blood components and exhaled breath.

### DETAILED DISCLOSURE

The present invention provides a method for detecting biological conditions through noninvasive analysis of bodily fluid samples, including exhaled breath and blood. The present invention also includes methods for screening those analytes/biomarkers and their concentrations likely to be present in exhaled breath. A focus of the present invention is on the detection of analytes/biomarkers in an individual's bodily fluids indicative of conditions or diseases such as intoxication, cancer, renal failure, liver disease, or diabetes.

It is advantageous to define several terms before describing the invention. It should be appreciated that the following definitions are used throughout this application.

Definitions

As used in the specification and in the claims, the singular form of “a,” “an,” and “the” may include plural referents unless the context clearly dictates otherwise. Also, as used in the specification and in the claims, the term “comprising” may include the embodiments “consisting of” and consisting essentially of.”

The term “molecular recognition agent,” as used herein, refers to any entity that exhibits high affinity for a target analyte/biomarker of the invention. In accordance with the subject invention, a molecular recognition agent can specifically bind to a target analyte through chemical, biochemical, and/or physical means. In certain embodiments, the molecular recognition agent(s) is attached to a surface of a nanotube.

Generally, according to the present invention, aptamers are one form of molecular recognition agent that are utilized to detect whether there exist certain analytes/biomarkers within a subject fluid sample. The term “aptamer,” as used herein, refers to an oligonucleotide chain that has a specific binding affinity for a target compound or molecule of interest. Aptamers include nucleic acids that are identified from a candidate mixture of nucleic acids.

The term “molecular beacon,” as used herein, refers to a particle that comprises a molecule, or group of molecules, that becomes detectable under preselected conditions (such a molecule is also referred to herein as a signaling agent) (e.g., an energy transfer complex, fluorophores, amplifying fluorescent polymers, or chromophore(s)), wherein the molecule(s) is conjugated to a molecular recognition agent, such as an aptamer.

As used herein, “biomarkers” refer to naturally occurring or synthetic compounds, which are a marker of a disease state or of a normal or pathologic process that occurs in an organism (i.e., drug metabolism). The term “analyte,” as used herein, refers to any substance, including chemical and biological agents such as nucleic acids, proteins, illicit drugs, explosives, toxins, pharmaceuticals, carcinogens, poisons, allergens, and infectious agents, that can be measured in an analytical procedure.

The term “bodily fluid,” as used herein, refers to a mixture of molecules obtained from a patient. Bodily fluids include, but are not limited to, exhaled breath, whole blood, blood plasma, urine, semen, saliva, lymph fluid, meningal fluid, amniotic fluid, glandular fluid, sputum, feces, sweat, mucous, and cerebrospinal fluid. Bodily fluid also includes experimentally separated fractions of all of the preceding solutions or mixtures containing homogenized solid material, such as feces, tissues, and biopsy samples.

According to the subject invention, biomarkers and/or analytes detectable in exhaled breath using the systems and methods of the invention include those that may be found in breath gas, breath condensate, respiratory droplet, breath evaporate, water vapor, and/or bronchial or alveolar aerosols.

The terms “volatile” or “surrogate” biomarker, as used herein, refer to a molecule or compound that is detectable by means of its physical or chemical properties as an indication that a target analyte/biomarker is present in a patient's body. Such volatile or “surrogate” biomarkers preferably include olfactory markers (odors) that are detectable in exhaled breath or by a number of sensor technologies including, for example, AFPs. Volatile or “surrogate” biomarkers can be detected using a method according to the subject invention or by devices and methods known in the art including, but not limited to, gas chromatography, electronic noses, spectrophotometers to detect the volatile biomarker's infrared (IF), ultraviolet (UV), or visible absorbance or fluorescence, or mass spectrometers to detect characteristic mass display of a “surrogate” biomarker.

The systems and methods of the invention comprise at least one molecular recognition agent and at least one signaling agent, wherein the molecular recognition agent(s) and signaling agent(s) together provide a means for detecting, signaling, and/or quantifying virtually any compound of interest in exhaled breath.

In another embodiment, the systems of the invention include aptamers in combination with nanotechnology (i.e., nanotubes) to provide an effective method for signaling the presence of a target analyte in bodily fluids, including but not limited to the exhaled breath and blood.

In one embodiment, the present invention provides a method for analyzing target analytes/biomarkers in exhaled breath using a volatile or “surrogate” biomarker as the signaling agent. For example, one method for analyzing analytes/biomarkers in bodily fluids comprises the use of an aptamer or other form of molecular recognition agent attached directly to a volatile or “surrogate” biomarker. Volatile or “surrogate” biomarkers include substances and compounds that can be detected in bodily fluids by various sensing means. In certain embodiments, the surrogate biomarkers become volatile after the molecular recognition agent binds to the specific/target biomarker.

In another embodiment, the present invention provides a method for analyzing analytes/biomarkers in exhaled breath using an aptamer or other form of molecular recognition agent attached to a volatile biomarker that contains an amplifying fluorescent polymer (AFP). In an alternate, related embodiment, the invention provides a method for analyzing analytes/biomarkers in exhaled breath using an aptamer or other molecular recognition agent attached directly to an AFP.

According to the subject invention, surrogate biomarkers, molecular beacons, and/or amplifying fluorescent polymers can be detected and/quantified using conventional sensing devices. For example, previously disclosed sensor technology such as, but not limited to, microgravimetric sensor technology, electrochemical sensor technology, gas chromatography/mass spectrometry sensor technology, infrared spectroscopy technology, ion mobility spectrometry, photo-ionization sensor technology, thickness-shear mode sensor technology, amplifying fluorescent polymer sensor technology, fiber optic microsphere sensor technology, semiconductor sensor technology, conductive polymer sensor technology, surface acoustic wave sensor technology, immunoassays, interdigitated microelectrode array sensor technology, microelectromechanical sensor technology, and molecularly imprinted polymeric film sensor technology, can be used in accordance with the subject invention to detect and/or quantify the target analytes/biomarkers in bodily fluids.

In another embodiment, the present invention provides a method for analyzing analytes/biomarkers in bodily fluids, including blood, using a biosensor that includes a nanotube and an aptamer or other molecular recognition agent. The nanotube comprises a hollow tubular body defining an inner void, comprising a first end and a second end, and a volatile or “surrogate” biomarker contained within the hollow tubular body. In a preferred embodiment, the first end of the tubular body is open and a first end cap bound to an aptamer or other molecular recognition agent is positioned over the first open end to close the first end. The second end of the tube is closed or similarly capped as the first end.

According to the present invention, nanotubes containing volatile or “surrogate” biomarkers are provided that release the volatile or “surrogate” biomarkers from the nanotube under a variety of conditions (such as binding to a target analyte/biomarker) to enable diagnosis and/or treatment of a disease associated with the target analyte/biomarker.

Biosensing exhaled breath utilizing methods disclosed herein can be applied to a wide range of point of care (POC) diagnostic tests. For example, potential applications include detection of licit and illicit drugs, detection of a wide range of biomarkers related to specific diseases, and detection of any other compounds that appear in blood or other bodily fluids (such as exhaled breath, in particular exhaled breath condensates). These tests can be highly quantitative with the quantity of volatile or “surrogate” biomarker released/detectable being proportional to the quantity of a target compound in a sample of bodily fluid. In addition, the methods of the present invention can also evaluate pharmacodynamics and pharmacokinetics for drug interventions in individuals.

Molecular Recognition Agents

According to the subject invention, molecular recognition agents possess the ability to bind the analyte/biomarker of interest and, in certain circumstances, also possess the ability to create a modulated signal.

In one embodiment, the molecular recognition agent is a molecular beacon comprising a receptor molecule that possesses the ability to bind to the analyte/biomarker of interest and to create a modulated signal. In a related embodiment, the molecular recognition agent is a receptor molecule conjugated to a signaling agent. The molecular recognition agent preferably possesses the ability to specifically bind to an analyte/biomarker of interest. Upon binding the analyte/biomarker of interest, the molecular recognition agent may cause the signaling agent to produce the modulated signal. According to the subject invention, molecular recognition agents are chosen for their ability to specifically bind to the analyte/biomarker molecules in a specific manner.

A variety of natural and synthetic molecular recognition agents may be used in accordance with the subject invention. A molecular recognition agent of the invention can be selected such that it interacts (such as binds) only with the target analyte/biomarker species to be detected. Molecular recognition agents that may be used include, but are not limited to, antibodies (including polyclonal and monoclonal antibodies), recombinant antibodies, chimeric antibodies, enzymes, antigens, recombinant antigens, chimeric antigens, carbohydrates, lectins, nucleotide sequences (including recombinant, and chimeric nucleotides), peptide sequences (including recombinant and chimeric peptide sequences), polymeric acids, polymeric bases, protein binders, peptide binders, chelating agents (such as a crown ether or cryptand), aptamers, biochemical or biological (such as cellular) ligands or receptors, DNA aptamers, RNA aptamers, synthetic receptors, or a combination thereof.

In one embodiment, a naturally occurring or synthetic molecular recognition agent (MRA) is bound to a polymeric bead. The MRA-polymeric bead, in some embodiments, is capable of both binding the analyte(s)/biomarker(s) of interest and creating a detectable signal. In some embodiments, the MRA-polymeric bead will create an optical (e.g., absorbance or reflectance) or fluorescence/phosphorescent signal when bound to an analyte/biomarker of interest.

According to the subject invention, molecular recognition agents may be attached to any surface of a nanotube or nanocap using known methods for chemical or physical attachment. In certain embodiments, molecular recognition agents are covalently attached to nanotube(s) or nanocap(s) via functional groups introduced by functionalization of the surface of the nanotube or nanocap. Alternatively, molecular recognition agents may be covalently attached to a nanotube or nanocap via linker molecules. Molecular recognition agents may also be attached to the nanotube or nanocap by non-covalent linkage, for example by absorption via hydrophobic binding or Van der Waals forces, hydrogen bonding, acid/base interactions and electrostatic forces.

In one embodiment, the molecular recognition agent of the present invention is an antibody specific to a target analyte/biomarker. An antibody has a recognized structure that includes an immunoglobulin heavy and light chain. The heavy and light chains include an N-terminal variable region (V) and a C-terminal constant region (C). The heavy chain variable region is often referred to as “V_{H}” and the light chain variable region is referred to as “V_{L}”. The V_{H} and V_{L} chains form a binding pocket that has been referred to as F(v). See generally Davis, 3: 537, Ann. Rev. of Immunology (1985); and Fundamental Immunology 3rd Ed., W. Paul Ed. Raven Press LTD. New York (1993). Such structures facilitate highly effective and specific binding of an antibody to a target analyte.

Alternatively, recombinant bispecific antibody (bsFv) molecules can be used as molecular recognition agents of the invention. In one embodiment, bsFv molecules that bind a T-cell protein termed “CD3” and a TAA are used as molecular recognition agents in accordance with the present invention.

Aptamer Technology

With certain embodiments of the present invention, the molecular recognition agent is in the form of an aptamer. The discovery of the SELEX™ (Systematic Evolution of Ligands by EXponential enrichment) methodology enabled the identification of aptamers that recognize molecules other than nucleic acids with high affinity and specificity (see, for example, Ellington and Szostak, “In vitro selection of RNA molecules that bind specific ligands,” Nature, 346:818-822 (1990); Gold et al., “Diversity of oligonucleotide functions,” Ann. Rev. Biochem., 64:763-797 (1995); Tuerk and Gold, “Systematic evolution of ligands by exponential enrichment—RNA ligands to bacteriophage-T4 DNA-polymerase,” Science, 249:505-510 (1990), all of which are herein incorporated in their entirety by reference). Aptamers have been selected to recognize a broad range of targets, including small organic molecules as well as large proteins (see Gold et al., supra.; Osborne and Ellington, “Nucleic acid selection and the challenge of combinatorial chemistry,” Chem. Rev., 97:349-370 (1997), which is herein incorporated by reference in its entirety).

The aptamers derived from the SELEX process, as described in U.S. Pat. No. 5,475,096; U.S. Pat. No. 5,270,163; and WO 91/19813 (all of which are incorporated by reference in their entirety), may be utilized in the present invention. These patents describe a method for making aptamers, each having a unique sequence and the property of binding specifically to a desired target compound or molecule. In certain embodiments, aptamers derived from the SELEX methodology may be utilized as molecular recognition agents in the present invention. The SELEX methodology is based on the insight that nucleic acids have sufficient capacity for forming a variety of two- and three-dimensional structures and sufficient chemical versatility available within their monomers to act as ligands (from specific binding pairs) with virtually any chemical compound, whether monomeric or polymeric. Molecules of any size or composition can serve as target analytes. See also Jayasena, S., “Aptamers: An Emerging Class of Molecules That Rival Antibodies for Diagnostics,” Clinical Chemistry, 45:9, 1628-1650 (1999), which is herein incorporated by reference in its entirety.

Aptamers that can be used in the present invention include those described in U.S. Pat. No. 5,656,739 (hereinafter the '739 patent, which is herein incorporated by reference in its entirety). The '739 patent describes nucleic acids as particularly useful assembly templates because they can be selected to specifically bind nonoligonucleotide target molecules with high affinity (e.g., Tuerk and Gold (1990), supra), and because they can hybridize by complementary base pairing. Both forms of recognition can be programmably synthesized in a single molecule or hybridized into a single discrete structure.

Aptamers can be attached to proteins utilizing methods well known in the art (see Brody, E. N. and L. Gold, “Aptamers as therapeutic and diagnostic agents,” J Biotechnol, 74(1):5-13 (2000) and Brody, E. N. et al., “The use of aptamers in large arrays for molecular diagnostics,” Mol Diagn, 4(4):381-8 (1999), both of which are herein incorporated by reference in their entirety). Such photo-cross-linkable aptamers allow for the covalent attachment of aptamers to proteins. More importantly, such aptamer-linked proteins can then be immobilized on a surface of a nanotube, nanocap, or polymeric bead.

For example, aptamer-linked proteins can be attached covalently to a nanotube, nanocap, or polymeric bead by functionalization of the surface of the nanotube, nanocap, or polymeric bead. Alternatively, aptamer-linked proteins can be covalently attached to a nanotube, nanocap, or polymeric bead surface via linker molecules. Non-covalent linkage provides another method for introducing aptamer-linked proteins to a nanotube, nanocap, or polymeric bead surface. For example, an aptamer-linked protein may be attached to a nanotube, nanocap, or polymeric bead surface by absorption via hydrophilic binding or Van der Waals forces, hydrogen bonding, acid/base interactions, and electrostatic forces.

Molecular Beacons and Amplifying Fluorescent Polymers

The present invention contemplates utilizing molecular beacons to non-invasively detect drugs, biomarkers, and other analytes in exhaled breath and other bodily fluids, such as blood. In one embodiment, molecular beacons are provided in which aptamers are conjugated to a signaling agent, wherein the signaling agent is detectable under preselected conditions (i.e., after aptamer binding to a an analyte/biomarker of interest).

In one embodiment of the present invention, signaling agents are attached to aptamers, wherein fluorescence resonance energy transfer (FRET) or non-fluorescence resonance energy transfer (non-FRET) interactions using the signaling agents provide detectable signals when in the presence of a particular analyte/biomarker of interest (see Stojanovic, M. et al., “Aptamer-Based Folding Fluorescent Sensor for Cocaine,” J. Am. Chem. Soc., 123:4928-4931 (2001), which is incorporated herein by reference in its entirety). The aptamer acts as a sensor to detect the presence of a specific target analyte/biomarker. Upon detection of the analyte/biomarker, the binding event of the aptamer to analyte(s)/biomarker(s) of interest cause the signaling agents to generate a detectable signal.

In one method for utilizing FRET (see, for example, U.S. Pat. No. 5,538,848, which is incorporated herein by reference in its entirety), a molecular beacon of the invention comprises a molecular recognition agent that is an aptamer (or nucleic acid sequence), which is labeled with signaling agents, a fluorescent reporter and a quencher moiety. The fluorescent reporter and quencher moiety are conjugated to the aptamer in a configuration that results in quenching of fluorescence in the unbound molecular beacon. When in the presence of an analyte/biomarker of interest, the aptamer sequence specifically hybridizes to the target analyte/biomarker. When hybridized, exonuclease activity of Taq polymerase degrades the aptamer thereby eliminating the intramolecular quenching maintained by the intact molecular beacon. Because the aptamer is designed to hybridize specifically to the target analyte/biomarker, the increase in fluorescence intensity of the sample, caused by enzymatic degradation of the aptamer, can be correlated with the concentration of analyte/biomarker present in the sample.

In another embodiment of the invention, a molecular beacon is constructed that comprises an aptamer sequence specific for a target analyte/biomarker, wherein the aptamer is embedded within two complementary arm sequences (see Tyagi et al, Nature Biotechnology, 14: at p. 303, col. 1, Ins. 22-30). To each termini of the aptamer sequence is attached one of either a fluorophore or quencher moiety. In the absence of the target analyte/biomarker, the arm sequences anneal to each other to thereby form a loop and hairpin stem structure which brings the fluorophore and quencher together. When contacted with target analyte/biomarker, the complementary aptamer sequence and target analyte/biomarker will hybridize. Because the hairpin stem cannot coexist with the rigid double helix that is formed upon hybridization, the resulting conformational change forces the arm sequences apart and causes the fluorophore and quencher to be separated. When the fluorophore and quencher are separated, energy of the donor fluorophore does not transfer to the acceptor moiety and the fluorescent signal is then detectable. Since unhybridized “Molecular Beacons” are non-fluorescent, it is not necessary that any excess molecular beacons be removed from an assay. In certain embodiments, the arm sequences of the molecular beacon constructs are unrelated to the aptamer sequence (See: Tyagi et al., Nature Biotechnology, 14: at p. 303, col. 1; In. 30, which is incorporated herein by reference in its entirety).

In one embodiment, signaling agents comprise a first fluorescent indicator and a second fluorescent indication, both of which are attached to a molecular recognition agent (such as an aptamer or receptor). When no analyte/biomarker of interest is present, short wavelength excitation excites the first fluorescent indicator to fluoresce. The short wavelength excitation, however, can cause little or no fluorescence of the second fluorescent indicator. After binding of the analyte/biomarker of interest to the molecular recognition agent, a structural change in the molecular recognition agent brings the first and second fluorescent indicators closer to each other. This change in the intermolecular distance allows for the excited first indicator to transfer a portion of its fluorescent energy to the second fluorescent indicator. The transfer of energy (FRET) can be measured by either a drop in the energy of the fluorescence of the first indicator molecule, or the detection of increased fluorescence by the second indicator molecule.

Alternatively, the first and second fluorescent indicators may initially be positioned such that short wavelength excitation, may cause fluorescence of both the first and second fluorescent indicators, as described above. After binding of a target analyte/biomarker to the receptor molecular recognition agent, a structural change in the molecular recognition agent causes the first and second fluorescent indicators to move further apart. This change in intermolecular distance inhibits the transfer of fluorescent energy from the first indicator to the second fluorescent indicator. This change in the transfer of energy can be measured by either a drop in energy of the fluorescence of the second indicator molecule, or the detection of increased fluorescence by the first indicator molecule.

According to the subject invention, fluorescent indicators can include energy transfer moieties comprising at least one energy donor and at least one energy acceptor moiety. Preferred fluorescent indicators include, but are not limited to, fluorophores such as fluorescein, derivatives of bodipy, 5-(2′-aminoethyl)-aminonaphthalene-1-sulfonic acid (EDANS), derivatives of rhodamine, Cy2, Cy3, Cy 3.5, Cy5, Cy5.5, Texas red and its derivatives. Though the previously listed fluorophores might also operate as acceptors, preferably, the acceptor moiety is a quencher moiety. Preferably, the quencher moiety is a non-fluorescent aromatic or heteroaromatic moiety. The preferred quencher moiety is 4-((-4-(dimethylamino)phenyl)azo) benzoic acid (dabcyl).

Labeling a molecular recognition agent is analogous to peptide labeling. Because the synthetic of assembly is essentially the same, any method commonly used to label a peptide may be used to label a molecular recognition agent, including an aptamer. Typically, the N-terminus of the polymer is labeled by reaction with a moiety having a carboxylic acid group or activated carboxylic acid group. One or more spacer moieties can optionally be introduced between the labeling moiety and the probing nucleobase sequence of the oligomer. Generally, the spacer moiety is incorporated prior to performing the labeling reaction. However, the spacer may be embedded within the label and thereby be incorporated during the labeling reaction. Methods for preparing labeled molecular recognition agents are well known to the skilled artisan and have been disclosed in various references, including, for example, U.S. Pat. No. 6,949,343, which is incorporated herein by reference in its entirety.

In another embodiment, an indicator ligand is preloaded onto the molecular recognition agent (MRA). A target analyte/biomarker can displace the indicator ligand to produce a change in the spectroscopic properties of the indicator ligand-MRA complex. In this case, the initial background absorbance is relatively large and decreases when the analyte/biomarker of interest is present.

The indicator ligand, in one embodiment, has a variety of spectroscopic properties that may be measured. These spectroscopic properties include, but are not limited to, ultraviolet absorption, visible absorption, infrared absorption, fluorescence, and magnetic resonance. In one embodiment, the indicator ligand is a dye having either a strong fluorescence, a strong ultraviolet absorption, a strong visible absorption, or a combination of these physical properties. Examples of indicator ligands include, but are not limited to, carboxyfluorescein, ethidium bromide, 7-dimethylamino-4-methylcoumarin, 7-diethylamino-4-methylcoumarin, eosin, erythrosin, fluorescein, Oregon Green 488, pyrene, Rhodamine Red, tetramethylrhodamine, Texas Red, Methyl Violet, Crystal Violet, Ethyl Violet, Malachite green, Methyl Green, Alizarin Red S, Methyl Red, Neutral Red, o-cresolsulfonephthalein, o-cresolphthalein, phenolphthalein, Acridine Orange, B-naphthol, coumarin, and α-naphthionic acid. When the indicator ligand is mixed with the molecular recognition agent, the molecular recognition agent and indicator ligand interact with each other such that the above mentioned spectroscopic properties of the indicator ligand, as well as other spectroscopic properties may be altered. The nature of this interaction may be a binding interaction, wherein the indicator ligand and molecular recognition agent (MRA) are attracted to each other with a sufficient force to allow the newly formed MRA-indicator ligand complex to function as a single unit. The binding of the indicator ligand and MRA to each other may take the form of a covalent bond, an ionic bond, a hydrogen bond, a van der Waals interaction, or a combination of these bonds.

Amplifying fluorescent polymers (AFPs) can be utilized in the present invention. An AFP is a polymer containing several chromophores that are linked together. As opposed to isolated chromophores that require 1:1 interaction with an analyte/biomarker of interest in conventional fluorescence detection, the fluorescence of many chromophores in an AFP can be influenced by a single molecule. For example, a single binding event to an AFP can quench the fluorescence of many polymer repeat units, resulting in an amplification of the quenching. Quenching is a process which decreases the intensity of the fluorescence emission.

Molecular beacons and AFPs, including their methods for preparation, that can be used in the present invention are described in numerous patents and publications, including U.S. Pat. No. 6,261,783 and Fisher, M. et al., “A Man-Portable Chemical Sniffer Utilizing Novel Fluorescent Polymers for Detection of Ultra-Trace Concentrations of Explosives Emanating from Landmines,” Paper from the 4^{th} International Symposium on “Technology and the Mine Problem” held at the Naval Postgraduate School in Monterey, Calif., on Mar. 12-16, 2000, Nomadics, Inc.

Nanotechnology

Nanoparticle-based delivery systems offer the potential for controlled release of a signal upon detection of a target analyte/biomarker in bodily fluids. The present invention provides a unique method for diagnosing a condition and/or disease in a patient by utilizing a nanoparticle-based biosensor that includes nanoparticles, aptamers, and volatile or “surrogate” biomarkers. Nanoparticles are preferably in the form of tubular bodies (“nanotubes”). Nanotubes can be produced in a wide range of sizes and composed of a wide range of materials, or combination of materials, optimized for in-vivo delivery. Preferably, nanotubes intended for in-vivo use are of a length less than 500 nm and a diameter less than 200 nm. Because of concerns over occlusion of blood flow at the microvasculature level, it is important not to make nanoparticles too large for intravenous applications.

A number of patents and publications describe nanotube technology. For example, U.S. Pat. No. 5,482,601 to Ohshima et al. describes a method for producing carbon nanotubes. Other methods for making and using nanotubes include the non-carbon nanotubes of Zettl et al., U.S. Pat. No. 6,063,243, the functionalized nanotubes of Fisher et al., U.S. Pat. No. 6,203,814, and the nanotubes of Martin et al., U.S. patent application Ser. No. 10/274,829, all of which are incorporated in their entirety by reference.

According to the present invention, the nanotube is hollow and has two ends, preferably wherein a first end is open and a second end is closed. The first open nanotube end can be blocked with an end cap so as to prevent the release of the contents within the hollow interior of the nanotube. In a preferred embodiment, an aptamer is attached to the end cap to block the first open end of the nanotube.

Suitable end caps used to block a nanotube opening include, for example, nanoparticles having a diameter slightly larger than the inside diameter of the nanotube, so as to occlude the open end of the nanotube. End caps are any piece of matter and can be composed of materials that are chemically or physically similar (or dissimilar) to the nanotube. The end cap can be a particle that has a maximum dimension of less than 100 μm. In a preferred embodiment, the end cap is of a spherical or spheroidal form. However, end caps of other shapes, including ellipsoidal, cylindrical, and irregular, can also be used.

As described herein, nanotubes can be prepared to include functionalized end caps with aptamers. A variety of methods are available to functionalize an end cap, depending on the composition of the end cap. For example, an end cap can be functionalized using well-known chemical methods such as those employed for polylactide synthesis. Functional groups (i.e., aptamers) can be introduced to functionalized end caps by copolymerization. Monomers derived from an amino acid or lactic acid can be synthesized using standard methods and then used for random copolymerization with lactide. Such functionalized end caps can allow for the application of aptamers to the end cap.

Aptamers can be attached to proteins utilizing methods well known in the art (see Brody, E. N. and L. Gold, “Aptamers as therapeutic and diagnostic agents,” J Biotechnol, 74(1):5-13 (2000) and Brody, E. N. et al., “The use of aptamers in large arrays for molecular diagnostics,” Mol Diagn, 4(4):381-8 (1999)). For example, photo-cross-linkable aptamers allow for the covalent attachment of aptamers to proteins. Such aptamer-linked proteins can then be immobilized on a functionalized end cap of a nanotube. For example, aptamer-linked proteins can be attached covalently to a nanotube end cap, including attachment of the aptamer-linked protein by functionalization of the end cap surface. Alternatively, aptamer-linked proteins can be covalently attached to an end cap via linker molecules. Non-covalent linkage provides another method for introducing aptamer-linked proteins to an end cap. For example, an aptamer-linked protein may be attached to an end cap by absorption via hydrophibic binding or Van der Waals forces, hydrogen bonding, acid/base interactions, and electrostatic forces.

Aptamer-attached end caps, according to the present invention, are bound to the nanotube until the detection of a target analyte/biomarker by the aptamer. End caps can be attached to nanotubes using a variety of methods. Methods for attaching an end cap to a nanotube are well-known to the skilled artisan and can include, but are not limited to, using: electrostatic attraction, hydrogen bonding, acid and/or basic sites located on the end cap/nanotube, covalent bonds, and other chemical linkages.

A volatile or “surrogate” biomarker is preferably present within the hollow interior of a nanotube. Upon detection of a target analyte/biomarker by an aptamer attached to an end cap, the volatile or “surrogate” biomarker can be released with the uncapping of the nanotube. The volatile or “surrogate” biomarker can then be detected using a method according to the subject invention or by devices and methods known in the art including, but not limited to, gas chromatography, electronic noses, spectrophotometers to detect the volatile biomarker's infrared (IF), ultraviolet (UV), or visible absorbance or fluorescence, or mass spectrometers to detect characteristic mass display of a “surrogate” biomarker. Preferable “surrogate” biomarkers include olfactory markers (odors) that are detectable in exhaled breath.

According to the present invention, a nanotube is designed to release its volatile or “surrogate” biomarker in the presence of a target analyte/biomarker. This is achieved by linking an aptamer specific to the target analyte/biomarker to the end cap of a nanotube to provide an “uncapping mechanism.” The uncapping mechanism is based upon the detection by the aptamer-end cap of surface markers on cell types (i.e., cancer cells), proteins in the blood (i.e., PSA for prostate cancer), or drugs in the body (i.e., illicit drugs or therapeutic drugs). The uncapping mechanism may require the use of energy-bearing biomolecular motors such as, but not limited to, the actin-based system (Dickinson, R. B. and D. L. Purich, “Clamped filament elongation model for actin-based motors,” Biophys J., 82:605-617 (2002)).

In one embodiment, the nanotube surface contains a first molecular recognition agent at or near at least one opening to the nanotube, wherein the first molecular recognition agent is highly specific for a compound that is not an analyte/biomarker of interest. The nanotube is exposed to a nanocap that contains a second molecular recognition agent that is weakly bound to the compound, wherein the second molecular recognition agent is highly specific for the analyte/biomarker of interest. The compound on the nanocap binds to the first molecular recognition agent, effectively causing the nanocap to block the opening of the nanotube. When the membrane is then exposed to a sample containing the target analyte/biomarker, the analyte/biomarker in the sample competes with the compound attached to the second molecular recognition agent. As a result of this competition, the target analyte/biomarker from the sample causes the displacement of the nanocap from the opening of the nanotube by binding to the second molecular recognition agent that held the nanocap in place. Since the analyte/biomarker of interest displaces the nanocap, the opening of the nanotube is effectively open to allow for the release of a surrogate marker (detection of the surrogate marker indirectly indicates presence and/or concentration of the target analyte).

Nanoparticle-based biosensors, according to the present invention, can be administered utilizing methods known to the skilled artisan. For example, nanoparticle-based biosensors can be administered intravenously, intradermlly, subcutaneously, orally or nasally (i.e., inhalation), transdermally (i.e., topical), transmucosally, and via the rectum.

Nanoparticle-based sensors for use in an organism can be prepared from biodegradable polymers and/or biocompatible polymers. As used herein, a “biodegradable” substance refers to a substance that can be decomposed by biological agents or by natural activity within an organism. Examples of contemplated biodegradable polymers include, but are not limited to: polyesters such as poly(caprolactone), poly(glycolic acid), poly(lactic acid), and polyhydroxybutrate; polyanhydrides such as poly(adipic anhydride) and poly(maleic anhydride); polydioxanone; polyamines; polyamides; polyurethanes; polyesteramides; polyorthoesters; polyacetals; polyketals; polycarbonates; polyorthocarbonates; polyphosphazenes; poly(malic acid); poly(amino acids); polyvinylpyrrolidone; poly(methyl vinyl ether); poly(alkylene oxalate); poly(alkylene succinate); polyhydroxycellulose; chitin; chitosan; and copolymers and mixtures thereof.

As used herein, a “biocompatible” substance includes those substances that are compatible with and have demonstrated no significant toxic effects on living organisms. Examples of contemplated biocompatible polymers include PLG (Poly(lactide-co-glycolide)), poly(ethylene glycol), and copolymers of poly(ethylene oxide) with poly(L-Lactic acid) or with poly(β-benzyl-L-aspartate). In a preferred embodiment, biocompatibility includes immunogenic compatability. An immunogenically compatible substance can include a substance that, when introduced into a body, does not significantly elicit humoral or cell-based immunity.

Further, a number of approaches can be used to make the surface of a nanoparticle-based biosensor according to the present invention both biocompatible and “stealthy.” For example, this can be accomplished by attaching a PEG-maleimide to the chain-end thiols on the outer surfaces of a nanoparticle. If the nanoparticle is in the shape of a tube and composed of gold or similar metals, the PEG chain can be attached by a thiol linker as described in Yu, S. et al., “Size-Based Protein Separations in Poly(ethylene glycol)-Derivatized Gold Nanotubule Membranes,” Nano Letters, 1, 495-498 (2001). Other examples of biocompatible polymers and surface treatments can be found in Majeti N. V. Ravi Kumar, “Nano and Microparticles as Controlled Drug Delivery Devices,” J. Pharm. Pharmaceut. Sci., 3(2):234-258 (2000).

The present invention provides methods for assessing the efficacy of interventions in real-time. For example, it is known that isoprostane levels increase in cerebral spinal fluid and blood after traumatic brain injury. Isoprostane may be readily detectable in exhaled breath. In accordance with the present invention, an aptamer-biosensor can be used to detect and measure isoprostane levels in patients who have suffered traumatic brain injury. By measuring isoprostane levels, a clinician can follow the course of the brain injury. In addition, a nanoparticle-based aptamer-biosensor can be incorporated into pharmaceutical compositions to treat traumatic brain injury. Moreover, by presenting an isoprostane specific aptamer-biosensor to exhaled breath in accordance with the present invention, it can be possible to evaluate the efficacy of interventions in real-time for treating traumatic brain injury. Accordingly, the method of the present invention can also evaluate pharmacodynamics and pharmacokinetics for drug interventions in individuals.

In an embodiment, a nanotube according to the present invention can detect the appearance of cancer antigens on the walls of cancer cells, cause uncapping which in turn releases a volatile or “surrogate” biomarker that can be readily detected in the breath, and thereby notify the patient or his/her physician that a cancer cell (s) was encountered in the patient's body.

Signaling Agents

As an indicator of the presence and/or concentration of a target analyte/biomarker, the signaling agent can be any compound that can be identified in bodily fluids including radiolabeled or fluorescent compounds, compounds that change the color of bodily fluids for detection by the naked eye, or compounds that are readily identified in bodily fluids using sensor technology.

Signaling agents of the invention include, but are not limited to, chromagens, chemiluminescers, radioactive labels, dyes that can be detected by optical absorbance, fluorophor molecules, quantum dots, redox-active molecules, as well as species that cause the pH of the solution to change raman-active substances. Chromagens include compounds which absorb light in a distinctive range so that a color may be observed, or emit light when irradiated with light of a particular wavelength or wavelength range (e.g., fluorescers). Examples of chromogens as signaling agents include, but are not limited to, colloidal particles (i.e., nanometer scale gold or magnetic-polymer composites); dyes (i.e., quinoline dyes, triarylmethane dyes, acridine dyes, alizarin dyes, phthaleins, insect dyes, azo dyes, anthraquinoid dyes, cyanine dyes, phenazathionium dyes, and phenazoxonium dyes, UV absorbers, IR absorbers, raman-active substances); and fluorescent compounds including, but not limited to fluorescent compounds having primary functionalities (i.e., 1- and 2-aminoaphthalene, p,p′-diaminostilbenes, pyrenes, quaternary phenanthridine salts, 9-aminoacridines, p,p′-diaminobenzophenone imines, anthracenes, oxacarbocyanine, merocyanine, 3-aminoequilenin, perylene, bis-benzoxazole, bis-p-oxazolyl benzene, 1,2-benzophenazin, retinol, bis-3-aminopyridinium salts, hellebrigenin, tetracycline, sterophenol, benzimidazolylphenylamine, 2-oxo-3-chromen, indole, xanthene, 7-hydroxycoumarin, phenoxazine, salicylate, strophanthidin, porphyrins, triarylmethanes and flavin) and fluorescent compounds having functionalities for linking or can be modified to incorporate such functionalities (i.e., dansyl chloride, fluoresceins such as 3,6-dihydroxy-9-phenylxanthhydrol, rhodamineisothiocyanate, N-phenyl 1-amino-8-sulfonatonaphthalene, N-phenyl 2-amino-6-sulfonatonaphthalene, 4-acetamido4-isothiocyanatostilbene-2,2′-disulfonic acid, pyrene-3-sulfonic acid, 2-toluidinonaphthalene-6-sulfonate, N-phenyl, N-methyl 2-aminonaphthalene-6-sulfonate, ethidium bromide, atebrine, auromine-0, 2-(9′-anthroyl)palmitate, dansyl phosphatidylethanolamine, N,N′-dioctadecyl oxacarbocyanine, N,N′-dihexyl oxacarbocyanine, merocyanine, 4-(3′-pyrenyl)butyrate, d-3-aminodesoxyequilenin, 12-(9′-anthroyl)stearate, 2-methylanthracene, 9-vinylanthracene, 2,2′-(vinylene-p-phenylene)-bis-benzoxazole, p-bis[2-(4-methyl-5-phenyloxazolyl)]benzene, 6-dimethylamino-1,2-benzophenazin, retinol, bis(3′-aminopyridinium) 1,10-decandiyl diiodide, sulfonaphthylhydrazone of hellebrigenin, chlortetra-cycline, N-(7-dimethylamino-4-methyl-2-oxo-3-chromenyl) maleimide, N-[p-(2-benzimidazolyl)-phenyl]maleimide, N-(4-fluoranthyl) maleimide, bis(homovanillic acid), resazarin, 4-chloro-7-nitro-2.1.3-benzooxadiazole, merocyanine 540, resorufin, rose bengal, and 2,4-diphenyl-3 (2H)-furanone).

A chemiluminescer involves a compound that becomes electronically excited by a chemical reaction and may then emit light which serves as the detectable signal or donates energy to a fluorescent acceptor. A diverse number of families of compounds have been found to provide chemiluminescence under a variety of conditions. One family of compounds is 2,3-dihydro-1,-4-phthalazinedione. The most popular compound is luminol, which is the 5-amino compound. Other members of the family include the 5 amino-6,7,8-trimethoxy- and the dimethylamino[ca]benz analog. These compounds can be made to luminesce with alkaline hydrogen peroxide or calcium hypochlorite and base.

Another family of chemiluminescence compounds is the 2,4,5-triphenylimidazoles, with lophine as the common name for the parent product. Chemiluminescent analogs include para-dimethylamino and -methoxy substituents. Chemiluminescence may also be obtained with oxalates, usually oxalyl active esters (e.g. p-nitrophenyl) and a peroxide (e.g., hydrogen peroxide), under basic conditions. Alternatively, luciferins may be used in conjunction with luciferase or lucigenins.

Various radioisotopes can be used to label compounds for use as signaling agents. Known radioactive labels include tritium (3H), radioactive iodine (¹²⁵I), radioactive carbon (¹⁴C), radioactive phosphorous (³²P), radioactive sulphur (³⁵S), radioactive calcium (⁴⁵Ca), radioactive chromium (⁵¹Cr), radioative ruthenium (¹⁰³Ru), radioactive iron (⁵⁹Fe), radioactive zinc (⁶⁵Zn), radioactive selenium (⁷⁵Se), or the like. Methods for labeling of compounds with radioactive labels are well known in the art.

In certain embodiments, the signaling agent is a benzodiazepine or benzodiazepine metabolite that is detectable in urine. Benzodiazepines and their metabolites readily pass through the renal system into urine making benzodiazepines and substances with similar properties especially suitable as compliance markers. Examples of benzodiazepines or benzodiazepine metabolites that can be used in the invention include diazepam and alprazolam.

Additional signaling agents contemplated herein include, without limitation, dimethyl sulfoxide (DMSO), acetaldehyde, acetophenone, anise, benzaldehyde, benzyl alcohol, benzyl cinnamate, cadinene, camphene, camphor, cinnamon, garlic, citronellal, cresol, cyclohexane, eucalyptol, and eugenol, eugenyl methyl ether. Such volatile markers are particularly advantageous for use in detection in exhaled breath.

The signaling agents of the invention also include additives that have been federally approved and categorized as GRAS (“generally recognized as safe”), which are available on a database maintained by the U.S. Food and Drug Administration Center for Food Safety and Applied Nutrition. Signaling agents categorized as GRAS and are readily detectable in bodily fluids include, and are not limited to, sodium bisulfate, dioctyl sodium sulfosuccinate, polyglycerol polyricinoleic acid, calcium casein peptone-calcium phosphate, botanicals (i.e., chrysanthemum; licorice; jellywort, honeysuckle; lophatherum, mulberry leaf; frangipani; selfheal; sophora flower bud), ferrous bisglycinate chelate, seaweed-derived calcium, DHASCO (docosahexaenoic acid-rich single-cell oil) and ARASCO (arachidonic acid-rich single-cell oil), fructooligosaccharide, trehalose, gamma cyclodextrin, phytosterol esters, gum arabic, potassium bisulfate, stearyl alcohol, erythritol, D-tagatose, and mycoprotein.

Sensor Technology

Sensor technology is used by the present invention to detect the presence of a signaling agent in a bodily fluid sample. The detection of a signaling agent signifies the presence and/or quantity of a target analyte/biomarker.

The following are examples of various sensor technologies that may be utilized in practicing the system and methods of the present invention.

A. Microgravimetric Sensors

Microgravimentric sensors are based on the preparation of polymeric- or biomolecule-based sorbents that are selectively predetermined for a particular substance, or group of structural analogs. A direct measurement of mass changes induced by binding of a sorbent with a target marker can be observed by the propagation of acoustic shear waves in the substrate of the sensor. Phase and velocity of the acoustic wave are influenced by the specific adsorption of target markers onto the sensor surface. Piezoelectric materials, such as quartz (SiO₂) or zinc oxide (ZnO), resonate mechanically at a specific ultrasonic frequency when excited in an oscillating field. Electromagnetic energy is converted into acoustic energy, whereby piezoelectricity is associated with the electrical polarization of materials with anisotropic crystal structure. Generally, the oscillation method is used to monitor acoustic wave operation. Specifically, the oscillation method measures the series resonant frequency of the resonating sensor. Types of sensors derived from microgravimetric sensors include quartz crystal microbalance (QCM) devices that apply a thickness-shear mode (TSM) and devices that apply surface acoustic wave (SAW) detection principle. Additional devices derived from microgravimetric sensors include the flexural plate wave (FPW), the shear horizontal acoustic plate (SH-APM), the surface transverse wave (STW) and the thin-rod acoustic wave (TRAW).

B. Conducting Polymers

Conducting polymer sensors promise fast response time, low cost, and good sensitivity and selectivity. The technology is relatively simple in concept. A conductive material, such as carbon, is homogeneously blended in a specific non-conducting polymer and deposited as a thin film on an aluminum oxide substrate. The films lie across two electrical leads, creating a chemoresistor. As the polymer is subjected to various chemical vapors, it expands, increasing the distance between carbon particles, and thereby increasing the resistance. The polymer matrix swells because analyte vapor absorbs into the film to an extent determined by the partition coefficient of the analyte. The partition coefficient defines the equilibrium distribution of an analyte between the vapor phase and the condensed phase at a specified temperature. Each individual detector element requires a minimum absorbed amount of analyte to cause a response noticeable above the baseline noise. Selectivity to different vapors is accomplished by changing the chemical composition of the polymer. This allows each sensor to be tailored to specific chemical vapors. Therefore, for most applications an array of orthogonal responding sensors is required to improve selectivity. Regardless of the number of sensors in the array, the information from them must be processed with pattern recognition software to correctly identify the chemical vapors of interest. Sensitivity concentration are reportedly good (tens of ppm). The technology is very portable (small and low power consumption), relatively fast in response time (less than 1 minute), low cost, and should be rugged and reliable

C. Electrochemical Sensors

Electrochemical sensors measure a change in output voltage of a sensing element caused by chemical interaction of a target marker on the sensing element. Certain electrochemical sensors are based on a transducer principle. For example, certain electrochemical sensors use ion-selective electrodes that include ion-selective membranes, which generate a charge separation between the sample and the sensor surface. Other electrochemical sensors use an electrode by itself as the surface as the complexation agent, where a change in the electrode potential relates to the concentration of the target marker. Further examples of electrochemical sensors are based on semiconductor technology for monitoring charges at the surface of an electrode that has been built up on a metal gate between the so-called source and drain electrodes. The surface potential varies with the target marker concentration.

Additional electrochemical sensor devices include amperometric, conductometric, and capacitive immunosensors. Amperometric immunosensors are designed to measure a current flow generated by an electrochemical reaction at a constant voltage. Generally, electrochemically active labels directly, or as products of an enzymatic reaction, are needed for an electrochemical reaction of a target marker at a sensing electrode. Any number of commonly available electrodes can be used in amperometric immunosensors, including oxygen and H₂O₂ electrodes.

Capacitive immunosensors are sensor-based transducers that measure the alteration of the electrical conductivity in a solution at a constant voltage, where alterations in conductivity are caused by biochemical enzymatic reactions, which specifically generate or consume ions. Capacitance changes are measured using an electrochemical system, in which a bioactive element is immobilized onto a pair of metal electrodes, such as gold or platinum electrodes.

Conductometric immunosensors are also sensor-based transducers that measure alteration of surface conductivity. As with capacitive immunosensors, bioactive elements are immobilized on the surface of electrodes. When the bioactive element interacts with a target marker, it causes a decrease in the conductivity between the electrodes.

Electrochemical sensors are excellent for detecting low parts-per-million concentrations. They are also rugged, draw little power, linear and do not require significant support electronics or vapor handling (pumps, valves, etc.) They are moderate in cost ($50 to $200 in low volumes) and small in size.

D. Gas Chromatography/Mass Spectrometry (GC/MS)

Gas Chromatography/Mass Spectrometry (GC/MS) is actually a combination of two technologies. One technology separates the chemical components (GC) while the other one detects them (MS). Technically, gas chromatography is the physical separation of two or more compounds based on their differential distribution between two phases, the mobile phase and stationary phase. The mobile phase is a carrier gas that moves a vaporized sample through a column coated with a stationary phase where separation takes place. When a separated sample component elutes from the column, a detector converts the column eluent to an electrical signal that is measured and recorded. The signal is recorded as a peak in the chromatogram plot. Chromatograph peaks can be identified from their corresponding retention times. The retention time is measured from the time of sample injection to the time of the peak maximum, and is unaffected by the presence of other sample components. Retention times can range from seconds to hours, depending on the column selected and the component. The height of the peak relates to the concentration of a component in the sample mixture.

After separation, the chemical components need to be detected. Mass spectrometry is one such detection method, which bombards the separated sample component molecules with an electron beam as they elute from the column. This causes the molecules to lose an electron and form ions with a positive charge. Some of the bonds holding the molecule together are broken in the process, and the resulting fragments may rearrange or break up further to form more stable fragments. A given compound will ionize, fragment, and rearrange reproducibly under a given set of conditions. This makes identification of the molecules possible. A mass spectrum is a plot showing the mass/charge ratio versus abundance data for ions from the sample molecule and its fragments. This ratio is normally equal to the mass for that fragment. The largest peak in the spectrum is the base peak. The GC/MS is accurate, selective and sensitive.

E. Infrared Spectroscopy (FTIR, NDIR)

Infrared (IR) spectroscopy is one of the most common spectroscopic techniques used by organic and inorganic chemists. Simply, it is the absorption measurement of different IR frequencies by a sample positioned in the path of an IR beam. IR radiation spans a wide section of the electromagnetic spectrum having wavelengths from 0.78 to 1000 micrometers (microns). Generally, IR absorption is represented by its wave number, which is the inverse of its wavelength times 10,000. For a given sample to be detected using IR spectroscopy, the sample molecule must be active in the IR region, meaning that the molecule must vibrate when exposed to IR radiation. Several reference books are available which contain this data, including the Handbook of Chemistry and Physics from the CRC Press.

There are two general classes of IR spectrometers—dispersive and non-dispersive. In a typical dispersive IR spectrometer, radiation from a broadband source passes through the sample and is dispersed by a monochromator into component frequencies. The beams then fall on a detector, typically a thermal or photon detector, which generates an electrical signal for analysis. Fourier Transform IR spectrometers (FTIR) have replaced the dispersive IR spectrometer due to their superior speed and sensitivity. FTIR eliminates the physical separation of optical component frequencies by using a moving mirror Michelson interferometer and taking the Fourier transform of the signal.

Conversely, in the non-dispersive IR (NDIR) spectrometer, instead of sourcing a broad IR spectrum for analyzing a range of sample gases, the NDIR sources a specific wavelength which corresponds to the absorption wavelength of the target sample. This is accomplished by utilizing a relatively broad IR source and using spectral filters to restrict the emission to the wavelength of interest. For example, NDIR is frequently used to measure carbon monoxide (CO), which absorbs IR energy at a wavelength of 4.67 microns. By carefully tuning the IR source and detector during design, a high volume production CO sensor is manufactured. This is particularly impressive, as carbon dioxide is a common interferent and has an IR absorption wavelength of 4.26 microns, which is very close to that of CO.

NDIR sensors promise low cost (less than $200), no recurring costs, good sensitivity and selectivity, no calibration and high reliability. They are small, draw little power and respond quickly (less than 1 minute). Warm up time is nominal (less than 5 minutes). Unfortunately, they only detect one target gas. To detect more gases additional spectral filters and detectors are required, as well as additional optics to direct the broadband IR source.

F. Ion Mobility Spectrometry (IMS)

Ion Mobility Spectrometry (IMS) separates ionized molecular samples on the basis of their transition times when subjected to an electric field in a tube. As the sample is drawn into the instrument, it is ionized by a weak radioactive source. The ionized molecules drift through the cell under the influence of an electric field. An electronic shutter grid allows periodic introduction of the ions into the drift tube where they separate based on charge, mass, and shape. Smaller ions move faster than larger ions through the drift tube and arrive at the detector sooner. The amplified current from the detector is measured as a function of time and a spectrum is generated. A microprocessor evaluates the spectrum for the target compound, and determines the concentration based on the peak height.

IMS is an extremely fast method and allows near real time analysis. It is also very sensitive, and should be able to measure all the analytes of interest. IMS is moderate in cost (several thousand dollars) and larger in size and power consumption.

G. Metal Oxide Semiconductor (MOS) Sensors

Metal Oxide Semiconductor (MOS) sensors utilize a semiconducting metal-oxide crystal, typically tin-oxide, as the sensing material. The metal-oxide crystal is heated to approximately 400° C., at which point the surface adsorbs oxygen. Donor electrons in the crystal transfer to the adsorbed oxygen, leaving a positive charge in the space charge region. Thus, a surface potential is formed, which increases the sensor's resistance. Exposing the sensor to deoxidizing, or reducing, gases removes the surface potential, which lowers the resistance. The end result is a sensor which changes its electrical resistance with exposure to deoxidizing gases. The change in resistance is approximately logarithmic.

MOS sensors have the advantage of being extremely low cost (less than $8 in low volume) with a fast analysis time (milliseconds to seconds). They have long operating lifetimes (greater than five years) with no reported shelf life issues.

H. Thickness-Shear Mode Sensors (TSM)

TSM sensors consist of an AT-cut piezoelectric crystal disc, most commonly of quartz because of its chemical stability in biological fluids and resistance to extreme temperatures, and two electrodes (preferably metal) attached to opposite sides of the disc. The electrodes apply the oscillating electric field. Generally, TSM sensor devices are run in a range of 5-20 MHz. Advantages are, besides the chemical inertness, the low cost of the devices and the reliable quality of the mass-produced quartz discs.

I. Photo-Ionization Detectors (PID)

Photo-Ionization Detectors rely on the fact that all elements and chemicals can be ionized. The energy required to displace an electron and ‘ionize’ a gas is called its Ionization Potential (IP), measured in electron volts (eV). A PID uses an ultraviolet (UV) light source to ionize the gas. The energy of the UV light source must be at least as great as the IP of the sample gas. For example, benzene has an IP of 9.24 eV, while carbon monoxide has an IP of 14.01 eV. For the PID to detect the benzene, the UV lamp must have at least 9.24 eV of energy. If the lamp has an energy of 15 eV, both the benzene and the carbon monoxide would be ionized. Once ionized, the detector measures the charge and converts the signal information into a displayed concentration. Unfortunately, the display does not differentiate between the two gases, and simply reads the total concentration of both summed together.

Three UV lamp energies are commonly available: 9.8, 10.6 and 11.7 eV. Some selectivity can be achieved by selecting the lowest energy lamp while still having enough energy to ionize the gases of interest. The largest group of compounds measured by a PID are the organics (compounds containing carbon), and they can typically be measured to parts per million (ppm) concentrations. PIDs do not measure any gases with an IP greater than 11.7 eV, such as nitrogen, oxygen, carbon dioxide and water vapor. The CRC Press Handbook of Chemistry and Physics includes a table listing the IPs for various gases.

PIDs are sensitive (low ppm), low cost, fast responding, portable detectors. They also consume little power.

J. Surface Acoustic Wave Sensors (SAW)

Surface Acoustic Wave (SAW) sensors are constructed with interdigitated metal electrodes fabricated on piezoelectric substrates both to generate and to detect surface acoustic waves. Surface acoustic waves are waves that have their maximum amplitude at the surface and whose energy is nearly all contained within 15 to 20 wavelengths of the surface. Because the amplitude is a maximum at the surface such devices are very surface sensitive. Normally, SAW devices are used as electronic bandpass filters in cell phones. They are hermetically packaged to insure that their performance will not change due to a substance contacting the surface of the SAW.

SAW chemical sensors take advantage of this surface sensitivity to function as sensors. To increase specificity for specific compounds, SAW devices are frequently coated with a thin polymer film that will affect the frequency and insertion loss of the device in a predictable and reproducible manner. Each sensor in a sensor array is coated with a different polymer and the number and type of polymer coating are selected based on the chemical to be detected. If the device with the polymer coating is then subjected to chemical vapors that absorb into the polymer material, then the frequency and insertion loss of the device will further change. It is this final change that allows the device to function as a chemical sensor.

If several SAW devices are each coated with a different polymer material, the response to a given chemical vapor will vary from device to device. The polymer films are normally chosen so that each will have a different chemical affinity for a variety of organic chemical classes, that is, hydrocarbon, alcohol, ketone, oxygenated, chlorinated, and nitrogenated. If the polymer films are properly chosen, each chemical vapor of interest will have a unique overall effect on the set of devices. SAW chemical sensors are useful in the range of organic compounds from hexane on the light, volatility extreme to semi-volatile compounds on the heavy, low volatility extreme.

Motors, pumps and valves are used to bring the sample into and through the array. The sensitivity of the system can be enhanced for low vapor concentrations by having the option of using a chemical preconcentrator before the array. In operation, the preconcentrator absorbs the test vapors for a period of time and is then heated to release the vapors over a much shorter time span thereby increasing the effective concentration of the vapor at the array. The system uses some type of drive and detection electronics for the array. An on board microprocessor is used to control the sequences of the system and provide the computational power to interpret and analyze data from the array.

SAW sensors are reasonably priced (less than $200) and have good sensitivity (tens of ppm) with very good selectivity. They are portable, robust and consume nominal power. They warm up in less than two minutes and require less than one minute for most analysis. They are typically not used in high accuracy quantitative applications, and thus require no calibration. SAW sensors do not drift over time, have a long operating life (greater than five years) and have no known shelf life issues. They are sensitive to moisture, but this is addressed with the use of a thermally desorbed concentrator and processing algorithms.

K. Amplifying Fluorescent Polymer Technology

Sensors can use fluorescent polymers that react with volatile chemicals as sensitive target marker detectors. Conventional fluorescence detection normally measures an increase or decrease in fluorescence intensity or an emission wavelength shift that occurs when a single molecule of the target marker interacts with an isolated chromophore, where the chromophore that interacts with the target marker is quenched; the remaining chromophores continue to fluoresce.

A variation of this approach is the “molecular wire” configuration, as described by Yang and Swager, J. Am. Chem. Soc., 120:5321-5322 (1998) and Cumming et al., IEEE Trans Geoscience and Remote Sensing, 39:1119-1128 (2001). In the molecular wire configuration, the absorption of a single photon of light by any chromophore will result in a chain reaction, quenching the fluorescence of many chromophores and amplifying the sensory response by several orders of magnitude. Sensors based on the molecular wire configuration have been assembled for detecting explosives (see Swager and Wosnick, MRS Bull, 27:446-450 (2002).

L Fiber Optic Microsphere Technology

Fiber optic microsphere technology is based upon an array of a plurality of microsphere sensors (beads), wherein each microsphere belongs to a discrete class that is associated with a target marker, that is placed on an optical substrate containing a plurality of micrometer-scale wells (see, for example, Michael et al., Anal Chem, 71:2192-2198 (1998); Dickinson et al., Anal Chem., 71:2192-2198 (1999); Albert and Walt, Anal Chem, 72:1947-1955 (2000); and Stitzel et al., Anal Chem, 73:5266-5271 (1001)). Each type of bead is encoded with a unique signature to identify the bead as well as its location. Upon exposure to a target marker, the beads respond to the target marker and their intensity and wavelength shifts are used to generate fluorescence response patterns, which are, in turn, compared to known patterns to identify the target marker.

M. Interdigitated Microelectrode Arrays (IME)

Interdigitated microelectrode arrays are based on the used of a transducer film that incorporates an ensemble of nanometer-sized metal particles, each coated by an organic monomolecular layer shell (see, for example, Wohltjen and Snow, Anal Chem, 70:2856-2859 (1998); and Jarvis et al., Proceedings of the 3^{rd} Intl Aviation Security Tech Symposium, Atlantic City, N.J., 639-647 (2001)). Such sensor devices are also known as metal-insulator-metal ensembles (MIME) because of the combination of a large group of colloidal-sized, conducting metal cores separated by thin insulating layers.

N. Microelectromechanical Systems (MEMS)

Sensor technology based on MEMS integrate mechanical elements, sensors, actuators, and electronics on a common silicon substrate for use in detecting target markers (see, for example, Pinnaduwage et al., Proceedings of 3^{rd} Intl Aviation Security Tech Symposium, Atlantic City, N.J., 602-615 (2001); and Lareau et al., Proceedings of 3^{rd} Intl Aviation Security Tech Symposium, Atlantic City, N.J., 332-339 (2001)).

One example of sensor technology based on MEMS is microcantilever sensors. Microcantilever sensors are hairlike, silicon-based devices that are at least 1,000 times more sensitive and smaller than currently used sensors. The working principle for most microcantilever sensors is based on a measurement of displacement. Specifically, in biosensor applications, the displacement of a cantilever-probe is related to the binding of molecules on the (activated) surface of the cantilever beam, and is used to compute the strength of these bonds, as well as the presence of specific reagents in the solution under consideration (Fritz, J. et al., “Translating biomolecular recognition into nanomechanics,” Science, 288:316-318 (2000); Raiteri, R. et al., “Sensing of biological substances based on the bending of microfabricated cantilevers,” Sensors and Actuators B, 61:213-217 (1999)). It is clear that the sensitivity of these devices strongly depends on the smallest detectable motion, which poses a constraint on the practically vs. theoretically achievable performance.

One example of microcantilever technology uses silicon cantilever beams (preferably a few hundred micrometers long and 1 μm thick) that are coated with a different sensor/detector layer (such as antibodies or aptamers). When exposed to a target marker, the cantilever surface absorbs the target marker, which leads to interfacial stress between the sensor and the absorbing layer that bends the cantilever. Each cantilever bends in a characteristic way typical for each target marker. From the magnitude of the cantilever's bending response as a function of time, a fingerprint pattern for each target marker can be obtained.

Microcantilever sensors are highly advantageous in that they can detect and measure relative humidity, temperature, pressure, flow, viscosity, sound, ultraviolet and infrared radiation, chemicals, and biomolecules such as DNA, proteins, and enzymes. Microcantilever sensors are rugged, reusable, and extremely sensitive, yet they cost little and consume little power. Another advantage in using the sensors is that they work in air, vacuum, or under liquid environments.

O. Molecularly Imprinted Polymeric Film

Molecular imprinting is a process of template-induced formation of specific molecular recognition sites (binding or catalytic) in a polymeric material where the template directs the positioning and orientation of the polymeric material's structural components by a self-assembling mechanism (see, for example, Olivier et al., Anal Bioanal Chem, 382:947-956 (2005); and Ersoz et al., Biosensors & Bioelectronics, 20:2197-2202 (2005)). The polymeric material can include organic polymers as well as inorganic silica gels. Molecularly imprinted polymers (MIPs) can be used in a variety of sensor platforms including, but not limited to, fluorescence spectroscopy; UV/Vis spectroscopy; infrared spectroscopy; surface plasmon resonance; checmiluminescent adsorbent assay; and reflectometric interference spectroscopy. Such approaches allow for the realization of highly efficient and sensitive target marker recognition.

Other types of chemical sensors known in the art use chemoselective coating applicable to the operation of the present invention. Examples of such chemical sensors include bulk acoustic wave (BAW) devices, plate acoustic wave devices, interdigitated microelectrode (IME) devices, optical waveguide (OW) devices, electrochemical sensors, and electrically conducting sensors.

In certain embodiments, the invention uses fluid sensor technology, such as commercial devices known as “artificial noses,” “electronic noses,” or “electronic tongues.” These devices are capable of qualitative and/or quantitative analysis of simple or complex gases, vapors, odors, liquids, or solutions. A number of patents and patent applications which describe fluid sensor technology include the following: U.S. Pat. Nos. 5,945,069; 5,918,257; 5,891,398; 5,830,412; 5,783,154; 5,756,879; 5,605,612; 5,252,292; 5,145,645; 5,071,770; 5,034,192; 4,938,928; and 4,992,244; and U.S. Patent Application No. 2001/0050228. Certain sensitive, commercial off-the-shelf electronic noses, such as those provided by Cyrano Sciences, Inc. (“CSI”) (i.e., CSI's portable Electronic Nose and CSI's Nose-Chip™ integrated circuit for odor-sensing—U.S. Pat. No. 5,945,069), can be used in the present invention to detect the presence of detectable signaling agents in bodily fluid samples.

In other, competitive binding immunoassays can be used to test a bodily fluid sample for the presence of signaling agents. Immunoassay tests generally include an absorbent, fibrous strip having one or more reagents incorporated at specific zones on the strip. The bodily fluid sample is deposited on the strip and by capillary action the sample will migrate along the strip, entering specific reagent zones in which a chemical reaction may take place. At least one reagent is included which manifests a detectable response, for example a color change, in the presence of a minimal amount of a signaling agent of interest. Patents that describe immunoassay technology include the following: U.S. Pat. Nos. 5,262,333 and 5,573,955.

Analytes/Biomarkers

Many types of important antigens on cell surfaces indicate the presence of a wide variety of disease states, ranging from cancer, inflammatory disorders, and infections to cardiovascular disease. Surface cell markers can help identify a diseased cell (i.e., malignancy) in two ways: 1) by being uniquely expressed (not ordinarily present on the surface in normal cells), or 2) by being expressed in a greatly altered density (i.e., marked overexpression of a surface cell marker). For example, in the case of blood malignancies such as lymphomas and leukemias, unique markers and clusters of surface markers can be used to accurately identify blood cancers. Accordingly, analytes/biomarkers of interest of the present invention can include, without limitation, surface markers that identify disease states, including those surface markers known to identify leukemias and lymphomas via immunophenotyping.

Examples of such analytes/biomarkers of interest include, and are not limited to, (1) T cell markers (CD2, CD3, CD4, CD5, CD7, and CD8); B cell markers (CD19 and CD20); myeloid/monocytic markers (CD13, CD14, CD15, and CD33); maturity status markers (CD34, HLA-DR, and CD10=CALLA) that form an acute leukemia surface antigen profile; (2) pan-T cell markers: CD2, CD3, CD5; CD4 (helper) and CD8 (suppressor); pan-B markers CD19 and CD20; CD5 and CD20 (co-expression frequently indicates neoplastic proliferations) that form a chronic lymphocytic leukemia (CLL) and lymphoma Profile; (3) hairy cell markers CD11c (complement receptor), CD25 (IL-2 receptor), CD103, prolymphocytic/hairy cell marker FMC-7; B-lymphoid marker CD23 (evaluated in relationship to CD5 expression for the different diagnosis of CLL vs. MCL) that aid in diagnosing Hairy Cell Leukemia (HCL), Prolymphocytic Leukemia (PLL), or Mantle Cell Lymphoma/Leukemia; and (4) CD1, CD15, and CD30 (Ki-1) that indicate anaplastic lymphoma and Hodgkin's Disease.

Additional analytes/biomarkers detectable using the systems and methods of the present invention include those that are located in body fluids and that are not attached to cells. Such analytes/biomarkers not only include those biomarkers that are primarily released by diseased cells but also entail therapeutic and/or illicit drugs that have been imbibed.

Examples of such analytes/biomarkers of interest include, and are not limited to, the following: Alpha Fetoprotein (AFP), which is a useful tumor marker for the diagnosis and management of hepatocellular carcinoma and non-seminomatous testicular cancer; Beta2-Microglubulin (b2-M), high concentrations of which indicate active disease, cell turnover, tumor presence; the presence of inflammatory diseases (i.e., rheumatoid arthritis, systemic lupus erythematosus, Sjögren syndrome, Crohn's disease); or be a secondary indication of various lymphoproliferative diseases (leukemia, lymphoma, and multiple myeloma); Beta Human Chorionic Gonadotropin (b HCG), which is a tumor marker for gestational trophoblastic diseases, germ cell tumors of the ovary or testis, and cancers of the breast, lung, pancreas, stomach, kidney, and brain and is very helpful in assessing the efficacy of therapy in patients with testicular tumors; Carbohydrate antigen 19-9 (CA19-9), which is not organ specific but is a marker for a variety of adenocarcinomas (pancreatic, gastric, and hepatobiliary); and CA 125, which is found in most serous, endometrioid and clear cells carcinomas of the ovary.

Given the arrival of new technologies such as differential screening of phage displayed libraries to identify highly novel cell surface markers specific to different types of malignancies (i.e., ovarian cancer), the utility of the systems and methods of the present invention to detect, notify, and monitor a wide variety of disease processes will markedly increase in the next decade.

Exemplary Method for Diagnosing Bronchogenic Carcinoma

In a preferred embodiment, an aptamer is designed for a biomarker of bronchogenic carcinoma. Bronchogenic carcinomas produce carcinoma metabolites that cause the occurrence of O-toluidine in exhaled breath. An aptamer can be designed using routine techniques that is specific for O-toluidine (O-toluidine-aptamer). The O-toluidine-aptamer can be linked with a molecular beacon, such as an AFP, to form an OT-biosensor. Upon exposing an OT-biosensor to exhaled breath suspected of containing O-toluidine, the O-toluidine-aptamer specifically binds to any O-toluidine present and causes the molecular beacon, such as AFP, to generate a signal. Thus, a time- and cost-efficient test for the presence of bronchogenic carcinoma is provided.

Exemplary Method for Diagnosing Prostate Cancer

In another preferred embodiment, an aptamer is designed for a biomarker of a specific cancer, i.e., prostate cancer. Prostate cancers produce a protein, prostate specific antigen (PSA). An aptamer can be designed, using routine techniques, that is specific for PSA (PSA-aptamer). The PSA-aptamer can be attached to an end cap that fits on the end of a nanotube. In a rapid test for the presence of prostate cancer, or a recurrence, the volatile or “surrogate” biomarker is released from the nanotube after PSA (the biomarker of interest) interacts with the PSA-aptamer and “uncaps” the nanotubes. Using any of a number of previously disclosed detector technologies, the volatile biomarker is detected in exhaled breath that indicates the presence of PSA in the blood.

Screening Method According to the Present Invention

The present invention provides methods for determining which analytes/biomarkers and their concentrations are likely to be detectable in exhaled breath. Human blood is preferably employed. In one embodiment, human blood free of potential target analytes/biomarkers is screened as a baseline/control. In another embodiment, target analytes/biomarkers are added to human blood that is subsequently screened. The target analytes/biomarkers are preferably added to human blood in concentrations likely to be found in vivo in blood. The human blood (with or without target analytes/biomarkers) is then placed in closed containers and incubated at body temperature.

After incubation, the concentration of the target analyte/compound is assessed in whole blood, plasma, in an ultrafiltrate, and in the headspace using conventional quantitative/analytic devices including, but not limited to, liquid chromatography-mass spectroscopy (LS-MS) or gas chromatography-mass spectroscopy (GC-MS). Theoretically, the amount of target analyte/biomarker present in the ultrafiltrate should be proportional to the concentration detectable in exhaled breath. Measuring the amount of target analyte/biomarker in the headspace can provide a more accurate assessment of target analyte/biomarker concentration in exhaled breath. In a preferred embodiment, human blood samples including known target analytes/biomarkers are placed in vials and incubated at 98° F. The concentration of the target analyte/biomarker likely to be present in exhaled breath is assessed by measuring the amount of target analyte/biomarker present in the headspace using GC-MS.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application

We claim:

1. A method for detecting a target analyte/biomarker in exhaled breath comprising:
a) exposing to the exhaled breath a molecular recognition agent capable of selectively binding to the target analyte/biomarker, wherein the molecular recognition agent is linked with a signaling agent; and
b) detecting a signal generated by the signaling agent.

2. The method according to claim 1, wherein the signaling agent is an amplifying fluorescent polymer.

3. The method according to claim 1, wherein the molecular recognition agent is an aptamer.

4. The method according to claim 1, wherein the target analyte/biomarker is a nucleic acid, a protein, an illicit drug, an explosive, a toxin, a pharmaceutical, a carcinogen, a poison, an allergen, or an infectious agent.

5. The method according to claim 1, wherein the target analyte/biomarker is selected from the group consisting of acetaldehyde, acetone, ammonia, CO, chloroform, dichlorobenzene, diethylamine, hydrogen, isoprene, methanethiol, methylethylketone, O-toluidine, pentane sulfides and sulfides, H₂S, MES, and Me₂S.

6. The method according to claim 1, wherein the aptamer is capable of binding to isoprostane.

7. The method according to claim 1, wherein the signal is detected using sensor technology selected from the group consisting of: metal-insulator-metal ensemble (MIME) sensors, cross-reactive optical microsensor arrays, fluorescent polymer films, surface enhanced raman spectroscopy (SERS), diode lasers, selected ion flow tubes, metal oxide sensors (MOS), bulk acoustic wave (BAW) sensors, colorimetric tubes, infrared spectroscopy, gas chromatography, semiconductive gas sensor technology; mass spectrometers, fluorescent spectrophotometers, conductive polymer gas sensor technology; aptamer sensor technology; amplifying fluorescent polymer (AFP) sensor technology; microcantilever technology; molecularly polymeric film technology; surface resonance arrays; microgravimetric sensors; thickness sheer mode sensors; or surface acoustic wave gas sensor technology.

8. A method for detecting a target analyte/biomarker in a bodily fluid comprising:
a) exposing to a bodily fluid a nanotube comprising a hollow interior, a first end, a second end, a signaling agent located within the hollow interior, and an end cap, wherein the first end is open and the second end is closed, the first end being blocked with the end cap to prevent the release of the signaling agent, wherein a molecular recognition agent capable of specifically binding to the target analyte/biomarker is attached to the end cap;
c) detecting the signaling agent when the end cap is displaced from the first end upon molecular recognition agent detection of the target analyte/biomarker.

9. The method according to claim 8, wherein the bodily fluid is blood.

10. The method according to claim 8, wherein the signaling agent is an olfactory marker that is detectable in the bodily fluid.

11. The method according to claim 8, wherein the signaling agent is detected using sensor technology selected from the group consisting of: metal-insulator-metal ensemble (MIME) sensors, cross-reactive optical microsensor arrays, fluorescent polymer films, surface enhanced raman spectroscopy (SERS), diode lasers, selected ion flow tubes, metal oxide sensors (MOS), bulk acoustic wave (BAW) sensors, colorimetric tubes, infrared spectroscopy, gas chromatography, semiconductive gas sensor technology; mass spectrometers, fluorescent spectrophotometers, conductive polymer gas sensor technology; aptamer sensor technology; amplifying fluorescent polymer (AFP) sensor technology; microcantilever technology; molecularly polymeric film technology; surface resonance arrays; microgravimetric sensors; thickness sheer mode sensors; or surface acoustic wave gas sensor technology.

12. The method according to claim 8, wherein the molecular recognition agent is an aptamer that is capable of binding to prostate specific antigen.

13. A method for screening target analytes/biomarkers likely to be present in exhaled breath comprising:
a) providing a human blood samples;
b) placing the human blood sample into a closed container, wherein a headspace is provided;
c) incubating the closed container;
d) separating the incubated human blood sample into whole blood, plasma, and an ultrafiltrate;
e) assessing whether a target analyte/biomarker is present in the headspace.

14. The screening method according to claim 13, wherein the human blood sample is free of the target analyte/biomarker.

15. The screening method according to claim 13, further comprising the step of adding the target analyte/biomarker to the human blood sample before incubation.

16. The screening method according to claim 15, wherein the target analyte/biomarker is added to the human blood sample in a concentration likely to be found in blood in vivo.

17. The screening method according to claim 13, wherein the closed container is incubated at human temperature.
